(19) United States
(12) Patent Application Publication
(10) Pub. No.: US 2025/0090664 A1
(43) Pub. Date: Mar. 20, 2025

(54) PIEZOELECTRIC SHEAR-THINNING MATERIAL COMPOSITIONS AND METHODS FOR USE

(71) Applicant: Boston Scientific Scimed, Inc., Maple Grove, MN (US)

(72) Inventors: Ehsan Jabbarzadeh, Columbia, SC (US); Sara Eslambolchi Moghadam, Santa Clara, CA (US); Karen Dubbin, Mountain View, CA (US)

(73) Assignee: Boston Scientific Scimed, Inc., Maple Grove, MN (US)

(21) Appl. No.: 18/706,134

(22) PCT Filed: Jul. 13, 2022
(86) PCT No.: PCT/US2022/036980

§ 371 Date: Apr. 30, 2024

Related U.S. Application Data
(60) Provisional application No. 63/243,342, filed on Sep. 13, 2021.

(51) Int. Cl. A61K 41/00; A61K 9/14; A61K 9/16; A61K 49/04

(52) U.S. Cl. CPC A61K 41/0028; A61K 9/14; A61K 9/1629; A61K 41/00; A61K 49/04; A61K 49/0438

(57) ABSTRACT

Methods and compositions are disclosed herein to define a suite of shear-thinning hydrogels exhibiting piezoelectric properties. The piezoelectric materials described can be injected percutaneously or via transcatheter vascular route into a target environment for the locoregional stimulation of cells or tissues using wireless impulses as actuation mechanisms. These external stimuli introduce either an electrical or mechanical response in the implanted piezoelectric materials for medical interventions including tumor ablation, drug delivery, electroporation, chemo-electroporation, neural stimulation, wound healing, cardiovascular applications and musculoskeletal pain management.

### CROSS-REFERENCE TO RELATED APPLICATIONS

This application claims priority to PCT International Application No. PCT/US2022/036980, filed on Jul. 13, 2022, entitled “PIEZOELECTRIC SHEAR-THINNING MATERIAL COMPOSITIONS AND METHODS FOR USE”, which claims priority to U.S. Provisional Patent Application No. 63/243,342 filed Sep. 13, 2021, the entire contents of which are incorporated herein by reference and relied upon.

### FIELD OF THE DISCLOSURE

The present disclosure relates to implantable bioelectronics and piezoelectric materials for treatment of medical pathologies.

### BACKGROUND

Implantable bioelectronics provide researchers the ability to interface and communicate with the human body. These systems, including devices like pacemakers, cardiac defibrillators, and electrodes, have been developed to either stimulate tissues and organs through external stimuli or gather information from patients for diagnostic purposes. Recent developments in implantable bioelectronics have included increased interest in neural stimulation for the treatment of neurodegenerative disorders and for the treatment of injuries that may result in maladies ranging from paralysis to dyskinesia. Further, these and other implantable bioelectronics have shown promise as methods for pain management in chronic pain scenarios. Electrode materials have also been used for the treatment of tumors, wherein tumor cell ablation is caused by delivery of electric current or mechanical energy to the lesion. Further treatments using electrode materials include externally triggered drug release applications and systemic drug delivery treatments that make cells more susceptible to drug infiltration via electroporation. In the case of tumor cells, this process can be referred to as chemo-electroporation.

While implantable bioelectronics offer solutions to a number of human conditions, many are not broadly implemented because of issues related to electrode implantation (e.g., requiring invasive surgery for electrode implantation), physical encumberment caused by external hardware (e.g., electrodes and stimulation control devices), or tissue-electrode stiffness mismatch. For implantable electrodes, invasive surgery is required to embed devices in their target location, which can involve long recovery times. Similarly, many metal-based electrodes face a matrix stiffness mismatch, which can lead to discomfort or scarring for the patient.

Externally applied electrodes, for instance, are uncomfortable for patients, both during application of the electrodes as well as during the treatment period, where visible wires and devices worn for extended periods of time can be an inconvenience. This discomfort becomes a greater concern for bioelectrode systems requiring a wired power source that is either implanted proximate the electrode, and which may require follow-up appointments for replacement, or is located extracorporeally but requires patients to carry the wired power source on their person during treatment.

Thus, there exists a need for biocompatible electrode materials that can by wirelessly powered and delivered in a minimally invasive manner to increase the clinical relevance of these types of treatment.

### SUMMARY

In embodiments, the present disclosure provides implantable bioelectronics.

In an embodiment, the present disclosure relates to a piezoelectric, shear-thinning composition, comprising piezoelectric nanoparticles, one or more polymers, and deionized water.

In an embodiment, the composition comprises about 0.1% to about 50% (w/w) of piezoelectric nanoparticles.

In an embodiment, the piezoelectric nanoparticles are selected from the groups consisting of synthetic (laponite) and natural (bentonite, kaolinite, montmorillonite-smectite) nanoclays, quartz, zinc oxide nanoparticles, aluminum nitride.

In an embodiment, the composition comprises about 0.5% to about 20% (w/w) of one or more polymers.

In an embodiment, the polymer is selected from the group consisting of gelatin, collagen, chitosan, silk, polytetrafluoroethylene (PTFE), polylactic acid (PLA), poly-(l)-lactic acid (PLLA), poly (d)-lactic acid (PLDA), cellulose, alginate, agarose, starch, polyvinylidene fluoride (PVDF), polyethylene glycol (PEG), lignin, keratin, and polyvinyl alcohol (PVA).

In an embodiment, the composition includes a contrast agent selected from the group consisting of tantalum, tungsten, and iohexol.

In an embodiment, the storage modulus (G′) of the composition is from about 1 kPa to about 40 kPa.

In an embodiment, following percutaneous or vascular administration of the composition to a patient in need thereof, the exposure of the administered composition to an external stimulus provides an induced voltage from the composition.

In an embodiment, the induced voltage of the composition is from about 0.01 V to about 10,000 V.

In an embodiment, the external stimulus is selected from ultrasound stimulation, radiofrequency stimulation and microwave stimulation.

In an embodiment, the exposure of the composition to ultrasound frequencies between about 20 kHz to about 20 MHz provides an induced voltage from the composition.

In an embodiment, the exposure of the composition to radiofrequency frequencies between about between 50 MHz to about 200 MHz provides an induced voltage from the composition.

In an embodiment, the exposure of the composition to microwave frequencies between about 300 MHz to about 300 GHz provides an induced voltage from the composition.

In an embodiment, the present disclosure relates to a plurality of piezoelectric microgels or microbeads, wherein the microgels or microbeads comprise piezoelectric nanoparticles, and one or more polymers.

In an embodiment, the average particle diameter of the microgels or microbeads are from about 50 microns to about 1000 microns.

In an embodiment, the microgels or microbeads comprise about 0.1% to about 50% (w/w) of piezoelectric nanoparticles.

In an embodiment, the piezoelectric nanoparticles are selected from the groups consisting of synthetic (laponite) and natural (bentonite, kaolinite, montmorillonite-smectite) nanoclays, quartz, zinc oxide nanoparticles, aluminum nitride.

In an embodiment, the microgels or microbeads comprise about 0.5% to about 20% (w/w) of one or more polymers.

In an embodiment, the polymer is selected from the group consisting of gelatin, collagen, chitosan, silk, polytetrafluoroethylene (PTFE), polylactic acid (PLA), poly-(l)-lactic acid (PLLA), poly (d)-lactic acid PLDA, cellulose, alginate, agarose, starch, polyvinylidene fluoride (PVDF), polyethylene glycol (PEG), lignin, keratin, and polyvinyl alcohol (PVA).

In an embodiment, the piezoelectric microgels or microbeads further comprise a contrast agent selected from the group consisting of tantalum, tungsten, and iohexol.

In an embodiment, following percutaneous or vascular administration of the microspheres or microbeads to a patient in need thereof, the exposure of the administered composition to an external stimulus provides an induced voltage from the microspheres or microbeads.

In an embodiment, the induced voltage of the microgels or microbeads is from about 0.01 V to about 10,000 V.

In an embodiment, the external stimulus is selected from ultrasound stimulation, radiofrequency stimulation and microwave stimulation.

In an embodiment, the exposure of the microspheres or microbeads to ultrasound frequencies between about 20 kHz to about 20 MHz provides an induced voltage from the microgels or microbeads.

In an embodiment, the exposure of the microgels or microbeads to radiofrequency frequencies between about between 50 MHz to about 200 MHz provides an induced voltage from the microgels or microbeads.

In an embodiment, the exposure of the microgels or microbeads to microwave frequencies between about between about 300 MHz to about 300 GHz provides an induced voltage from the microgels or microbeads.

In an embodiment, the present disclosure relates to a method of treating cancer or a cancerous lesion through ablation, the method comprising (a) administering a therapeutically effective amount of the compositions described herein or the microgels or microbeads described herein, and (b) administering an external stimulus to provide an induced voltage from the composition, microgels, or microbeads.

In an embodiment, the composition, microgels, or microbeads are administered by transcatheter delivery or percutaneous injection.

In an embodiment, the external stimulus comprises applying sonic energy from an ultrasound or high-intensity focused ultrasound to the area where the composition, microgels, or microbeads is administered.

In an embodiment, the present disclosure relates to a method of neurostimulation, the method comprising (a) administering a therapeutically effective amount of the composition described herein or the microgels or microbeads described herein, and (b) administering an external stimulus to provide an induced voltage from the composition, microgels, or microbeads.

In an embodiment, the present disclosure relates to a method of pain management, the method comprising (a) administering a therapeutically effective amount of the composition described herein or the microgels or microbeads described herein and (b) administering an external stimulus to provide an induced voltage from the composition, microgels, or microbeads.

In an embodiment, the present disclosure relates to a method of enhancing wound healing, the method comprising (a) administering a therapeutically effective amount of the composition described herein or the microgels or microbeads described herein and (b) administering an external stimulus to provide an induced voltage from the composition, microgels, or microbeads.

In an embodiment, the present disclosure relates to a method of cardiovascular pacing, the method comprising (a) administering a therapeutically effective amount of the composition described herein or the microgels or microbeads described herein and (b) administering an external stimulus to provide an induced voltage from the composition, microgels, or microbeads.

In an embodiment, the present disclosure relates to a method of electroporation, either alone or in conjunction with chemotherapeutics, the method comprising (a) administering a therapeutically effective amount of the composition described herein or the microgels or microbeads described herein and (b) administering an external stimulus to provide an induced voltage from the composition, microgels, or microbeads.

In an embodiment, the external stimulus stimulates controlled release of encapsulated therapeutic agents, including chemotherapeutic agents.

### BRIEF DESCRIPTION OF THE SEVERAL VIEWS OF THE DRAWINGS

FIG. 1A provides a flow diagram of a method for delivering a piezoelectric-based bioelectronic, according to an exemplary embodiment of the present disclosure;

FIG. 1B depicts implementations of a piezoelectric-based bioelectronic, according to an exemplary embodiment of the present disclosure;

FIG. 2 is a graphical illustration of piezoelectric effect in three compositions of a piezoelectric-based bioelectronic comprising laponite, gelatin, and water, according to an exemplary embodiment of the present disclosure;

FIG. 3 is an illustration of three compositions of a piezoelectric-based bioelectronic based on laponite, gelatin, and water, according to an exemplary embodiment of the present disclosure;

FIG. 4 is an illustration of three compositions of a piezoelectric-based bioelectronic comprising varying amounts of tantalum contrast agent, according to an exemplary embodiment of the present disclosure; and

FIG. 5 is a graphical illustration of piezoelectric effect in compositions with varying amounts of tantalum contrast agent, according to an exemplary embodiment of the present disclosure.

### DETAILED DESCRIPTION

The term “a” or “an” refers to one or more of that entity, i.e. can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

Unlike traditional implantable devices intended to provide electrical stimulation or other therapeutic and/or diagnostic value to patients, the present disclosure describes methods by which a device, composition, and/or material, delivered in a minimally invasive manner so as to increase clinical relevance, may be electrically stimulated. Such a device is described herein, interchangeably, as a ‘bioelectronic’ device comprised of a ‘bioelectronic’ composition or ‘bioelectronic’ material.

One method by which electrical stimulation can be generated is using piezoelectric materials. Piezoelectric materials are a class of materials that respond to mechanical stimuli (e.g. ultrasonic pressure) with electrical charge or current. When energy sources such as radiofrequency or microwave are applied, the material is mechanically deformed. This phenomenon can be harnessed by tuning the frequency and amplitude of the energy source stimulating the piezoelectric material, which results in electrical or mechanical output directly at site of the material. Previously, piezoelectric materials have been utilized as a means to wirelessly trigger electronics through external stimuli such as ultrasound, radiofrequency, microwave, or other tissue-penetrating stimuli. This quality has been shown to result from the lack of symmetry in the central unit cell of piezoelectric crystals, resulting in uneven charge distribution in response to mechanical strain. Unfortunately, many of the most commonly used piezoelectric materials (e.g. lead zirconate titanate) are excluded from a healthcare setting as a result of toxic byproducts generated by the production or degradation of the piezoelectric material. Moreover, industrial piezoelectric materials also face size limitations, biological compatibility, and flexibility issues when being translated to biological applications. This has led to increased research into lead-free piezoelectric materials including polymer, composite, and nanostructured materials for piezoelectric-based bioelectronic devices.

Hydrogels provide an appealing alternative to conventional metal electrode materials for bioelectronics due to their viscoelastic nature. Hydrogel electrodes, therefore, can more closely integrate with the target tissue and mitigate issues associated with mismatch. Typically, hydrogels are soft materials with storage moduli within the range of human tissues and organs (<100 kPa), thereby avoiding issues with stiffness-induced scarring that are observed with metal electrodes. Piezoelectric hydrogels utilize the hierarchical and/or chiral structure of natural or engineered biopolymers to form structures with low symmetry, thereby resulting in a piezoelectric effect. Though biologically harvested materials such as proteins can show a piezoelectric effect, many of these biomaterials undergo chemical crosslinking that limit their tunability and injectability. As a result, the ability to deliver them via minimally invasive approaches is hindered. Thus, the compositions and/or materials described in the present disclosure, as will be detailed below, provide a clear advantage over previous methods.

### Compositions of the Present Disclosure:

In embodiments, the present disclosure provides bioelectronic compositions for the creation of injectable bioelectronics, thereby allowing for minimally invasive medical interventions. The bioelectronic composition of the present disclosure may be a bioelectronic shear thinning hydrogel. The bioelectronic composition of the present disclosure uses the piezoelectric effect as a means to be wirelessly energized through the application of external stimuli, such as ultrasound, radiofrequency, microwave, or other tissue-penetrating stimuli.

The bioelectronic composition may be a mixture of a piezoelectric substance, a carrier, and a solvent. The mixture could include, for example, a range of piezoelectric substances with concentrations between 0.1% to 50%, a range of carrier with concentrations between 0.5% to 20%, and the solvent as the balance.

In embodiments, the piezoelectric substance can be, among others, laponite, a charged nanosilicate, quartz, a zinc oxide nanoparticle, and aluminum nitride. In embodiments, the carrier can be a hydrogel. In embodiments, the carrier can be at least one polymer such as gelatin, collagen, chitosan, silk, polytetrafluoroethylene (PTFE), polylactic acid (PLA), poly-(l)-lactic acid (PLLA), poly (d)-lactic acid (PLDA), cellulose, alginate, agarose, starch, polyvinylidene fluoride (PVDF), polyethylene glycol (PEG), lignin, keratin, and polyvinyl alcohol (PVA), among others. In an example, the carrier is a gelatin hydrogel.

In embodiments, the composition may further include a contrast agent such as tantalum, tungsten, iohexol, omnipaque, or similar agent.

According to an embodiment, the bioelectronic composition is a shear-thinning composition. Shear-thinning is a non-Newtonian behavior of fluids whose viscosity decreases under strain. In other words, as certain forces (i.e., shear) are applied to such shear-thinning fluids, the fluids more readily flow. This allows the shear-thinning compositions of the present disclosure to be delivered via catheter, percutaneously, and the like.

Moreover, the bioelectronic composition may have mechanical properties similar to that of tissue proximate the bioelectronic composition upon implantation. For instance, a storage modulus (G′) of the bioelectronic composition may be between 1 kPa to 1 MPa. In embodiments, the storage modulus (G′) of the bioelectronic composition may be between 1 kPa and 100 kPa. In embodiments, the storage modulus (G′) of the bioelectronic composition is between 1 kPa and 40 kPa. As can be appreciated, the mechanical properties of the bioelectronic composition are dictated, in part, by the anticipated mechanical properties of tissues expected to be proximate the implanted bioelectronic composition.

In embodiments, the yield stress of the bioelectronic composition is from about 1 Pa to about 200 Pa. In some embodiments, the yield stress of the bioelectronic composition is from about 1 Pa to about 100 Pa. In embodiments, the yield stress of the bioelectronic composition is from about 2 Pa to about 50 Pa. In embodiments, the yield stress of the bioelectronic composition is from about 1 Pa to about 25 Pa. In embodiments, the yield stress of the bioelectronic composition is from about 1 Pa to about 10 Pa. In embodiments, the yield stress of the bioelectronic composition is from about 1 Pa to about 5 Pa. In embodiments, the bioelectronic composition flows upon application of a pressure greater than the yield stress.

In an embodiments, the phase transitioning qualities of the bioelectronic composition are determined by, among other things, ratios of ingredients within the bioelectronic composition and/or total solid content of the bioelectronic composition. The ratios of ingredients (e.g., oppositely charged polymers and nanoparticles) can impact electrostatic interactions. Together, the ratios of ingredients and total solid content determine viscoelastic properties (e.g., how the viscosity changes under shear rate and the extent of recovery/reversibility) of the bioelectronic composition.

In embodiments, size and shape of the bioelectronic composition can be determined by a particular mixture of, among other constituents, the piezoelectric substance, the carrier, and the solvent in view of the mechanical properties of the bioelectronic composition and its phase transitioning qualities. In low shear rates, the composition acts as a soft solid, and above a respective shear rate threshold, the composition acts as a viscous fluid. Such shear-thinning behavior allows the bioelectronic composition to be injectable. The solid may be a predetermined shape or may take the shape of the space in which it resides (i.e., implantation space). In some embodiments, the bioelectronic composition may be defined by a volume of the bioelectronic composition introduced, appreciating that the size and shape of the bioelectronic composition can vary based on application. In some embodiments, the bioelectronic composition may be configured as a microgel or a microbead. The microgel or the microbead may have the same composition as a larger bioelectronic composition, or may be different in composition (e.g., excluding a solvent or adding an additional constituent) but may have a size between 50 μm and 1000 μm in diameter. Reduced dimensions of the microgel or the microbead allow for penetration into capillaries having diameters ranging from between 100 μm to 5 mm.

### Method of Using the Compositions of the Present Disclosure:

Embodiments of the present disclosure provide, as the bioelectronic composition, a class of injectable, piezoelectric, shear thinning hydrogels for minimally invasive medical interventions. The bioelectronic composition, sometimes referred to herein as a bioelectronic material, can be implanted by a variety of means, including via direct percutaneous injection or via transcatheter vascular route. In an example, the bioelectronic composition can be delivered via direct percutaneous injection into a lesion (e.g., tumor). In an example, the bioelectronic composition is delivered via transcatheter vascular route (e.g., as an embolic material).

Embodiments of the present disclosure describe the formation of piezoelectric-microgels or -microbeads, based on the bioelectronic composition, to be delivered to the treatment site either via catheter or percutaneously through direct injection. Such approach allows for deeper penetration.

A bioelectronic device, based at least on the bioelectronic compositions defined herein, can be delivered directly to the treatment site through transcatheter delivery or percutaneous injection. When the bioelectronic device is delivered to the target area, it can be stimulated to induce electric current in the treated tissue through ultrasound, radiofrequency, microwave, or other energy sources. An externally located transducer can be used to deliver ultrasound, radiofrequency, microwave, or other energy field to the piezoelectric embolic material.

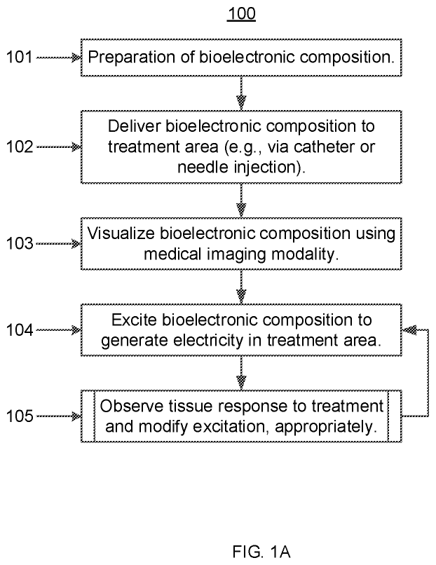

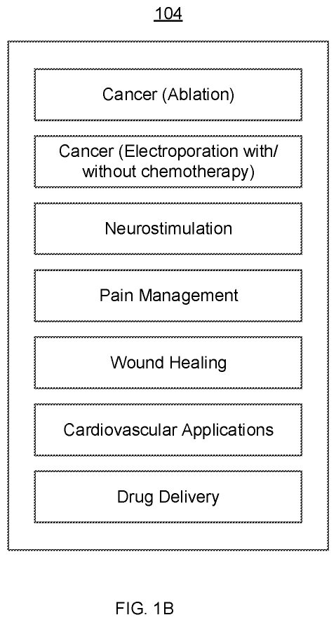

FIG. 1A provides a flow diagram of a method for delivering a bioelectronic device, a bioelectronic composition, and/or a bioelectronic material, according to an exemplary embodiment of the present disclosure. For clarity, the method will be described below with reference to a bioelectronic composition.

FIG. 1A is a flow diagram of a method 100 according to an embodiment in which a bioelectronic composition is delivered for treatment. At step 101 of method 100, the bioelectronic composition is prepared. In embodiments, the bioelectronic composition can be mixed such that the piezoelectric substance is uniformly suspended within the carrier. This allows the mixture to be used to produce a known quantity of charge or motion when activated by mechanical or electrical impulse. Various techniques can be used to achieve this uniform suspension. In embodiments, mechanical agitation (e.g., shaking and mixing) is used to create this uniform suspension. In other examples, a centrifuge or vortex agitator is used.

In embodiments, the bioelectronic composition prepared at step 101 of method 100 can be configured for a range of applications, as shown in FIG. 1B, and dimensional constraints, including for use as a microgel or a microbead. In embodiments, the bioelectronic composition can be configured to be delivered within lumens ranging from 50 microns to 10 millimeters in diameter.

At step 102 of method 100, the bioelectronic composition can be delivered to the treatment area. In embodiments, the bioelectronic composition can be delivered via a delivery device such as a needle or catheter configure to deliver the mixture to the treatment site. In embodiments, a needle can be advanced to the treatment site (e.g., percutaneous injection), and alternatively, a catheter is routed through the vasculature to a vein or artery at a vascular inflow to a treatment site.

At step 103 of method 100, a medical imaging modality such as magnetic resonance imaging (MRI), fluoroscopy, or ultrasound can be used to determine a position of the delivery device and/or the bioelectronic composition (in the event a contrast agent is included in the bioelectronic composition) during advancement or injection at step 102 of method 100. Once positioned accurately, the mixture can be injected to the treatment area. During and after injection, local deposition of the mixture in, for example, a tumor bed, can be identified. In the absence of contrast agent within the bioelectronic composition, the piezoelectric substance injected as part of the bioelectronic composition can be used to identify the local depositions based on feedback produced by the piezoelectric substance in response to electrical or mechanical stimuli.

At step 104 of method 100, the piezoelectric substance within the bioelectronic composition is excited in order to generate heat (by application of electrical impulse such as RF impulse) or electricity (by application of mechanical impulse such as ultrasound). Accurate location of the bioelectronic composition in the treatment area allows the applied energy for excitation to only impact a local area. In this way, an ultrasonic impulse can be delivered to the bioelectronic composition in order to generate electricity and ablate a region proximate where the bioelectronic composition was delivered without, in and of itself, damaging tissues. Likewise, an electromagnetic impulse such as an RF impulse could be delivered in order to cause mechanical deformations in the piezoelectric substance, and ablate tissues by heating, without causing damage to the tissue on its own.

In embodiments, the bioelectronic composition (ie., piezoelectric substances therein) is excited in accordance with a particular clinical outcome being sought. For instance, in the event of a solid cancer tumor, the clinical outcome may be ablation of the tumor. In embodiments, electroporation with or without chemotherapy for the treatment of cancer may be realized by excitation of the piezoelectric substance within the bioelectronic composition. In embodiments, excitation of the piezoelectric substance within the bioelectronic composition may allow for neurostimulation, pain management, wound healing, cardiovascular applications, and drug delivery, among others. Such implementations of method 100 will be described in greater detail throughout the remainder of the disclosure.

In embodiments, ultrasound frequencies between 20 kHz and 20 MHz is applied externally, corresponding to typically available therapeutic ultrasounds. Ultrasound intensities between 1000 W/m² to 100000 W/m² may also be used. For radiofrequency stimulation, frequencies between 50 MHz and 200 MHz may be used, corresponding to the range of frequencies categorized as radiofrequency. For microwave stimulation, frequencies between 300 MHz and 300 GHz may be utilized.

In embodiments, application of the stimulus, in the time-domain, will be based on a given implementation thereof. In other words, different applications of the methods described herein will benefit from different exposures to stimulus. As an example, the stimulus may be a short pulse, a long pulse, or a combination thereof. Of course, such examples are should not be considered limiting and are determined according to specific desired outcomes.

For external stimulation of the bioelectronic composition, an external stimulation device can be utilized for near-skin stimulation (<1 cm away from external stimulation) or deeper-skin stimulation (up to 15 cm), depending on the frequency corresponding to maximum penetration depths. This allows the bioelectronic composition to be stimulated when between about 0.01 cm up to about 25 cm from the surface of the skin.

In embodiments, external stimulation can induce voltages within the bioelectronic compositions of between 0.1 V and 10000 V. In embodiments, the bioelectronic compositions are excited between 2 and 100 times and may be configured to remain in the body for a minimum of 24 hours post-delivery. Stimulation, as it relates to number of excitations and resident time in the body can be determined according to requirements of specific implementations of the methods of the present disclosure. In an embodiment, the bioelectronic compositions can be biocompatible, bioresorbable, or a combination thereof. The resorption of the bioelectronic composition may be tailored to a specific application in order to provide therapeutic effect for a desired time period.

At 105 of method 100, excitation of the bioelectronic composition is monitored. Steps 104 and 105 of method 100 may be iterated until sufficient results are achieved. Such sufficient results can be determined by a medical professional, in an example. The sufficient result may be a clinical outcome related to ablation of a tissue.

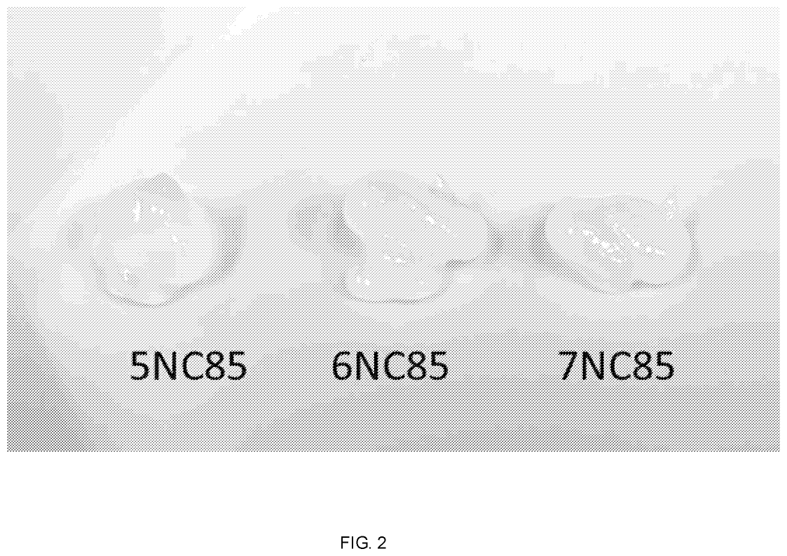

FIG. 2 provides illustrations of exemplary bioelectronic compositions, according to an embodiment of the present disclosure. In FIG. 2, the bioelectronic compositions include varying amounts of laponite (i.e., synthetic nanoclay), gelatin, and water. 5NC85 corresponds to 5 weight percent solid where 85% of total solid is nanoclay, 6NC85 corresponds to 6 weight percent solid where 85% of total solid is nanoclay, and 7NC85 corresponds to 7 weight percent solid where 85% of total solid is nanoclay.

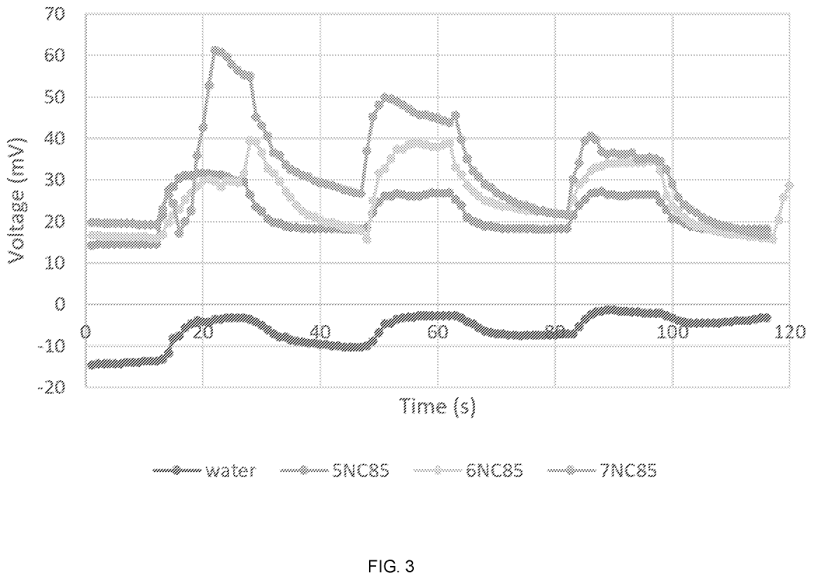

FIG. 3 is a graphical illustration of three bioelectronic compositions with varying amounts of laponite, gelatin, and water. Voltage was generated within each bioelectronic composition through application of 20 kHz sonic energy using a commercial sonic dismembrator. Excitations were applied in 15 second pulses every 20 seconds. Results indicate that voltage measured within each bioelectronic composition increased during periods when the sonic energy was applied.

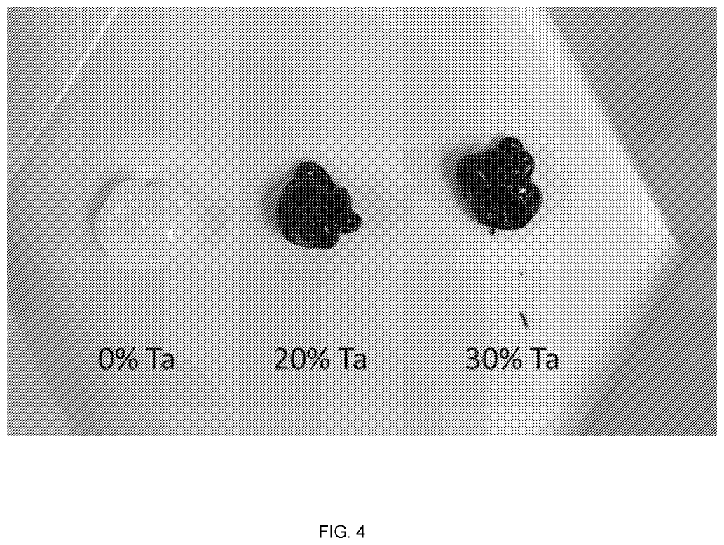

FIG. 4 provides illustrations of exemplary bioelectronic compositions, according to an embodiment of the present disclosure. In FIG. 4, the bioelectronic compositions include varying amounts of laponite, gelatin, water, and tantalum as a contrast agent. The doped nanoclay composition is 7NC85, corresponding to 7 weight percent solid where 85% of total solid is nanoclay. As can be appreciated from left to right in the image, tantalum was increased from 0% to 20% to 30%, and the color of each bioelectronic composition roughly reflects this change.

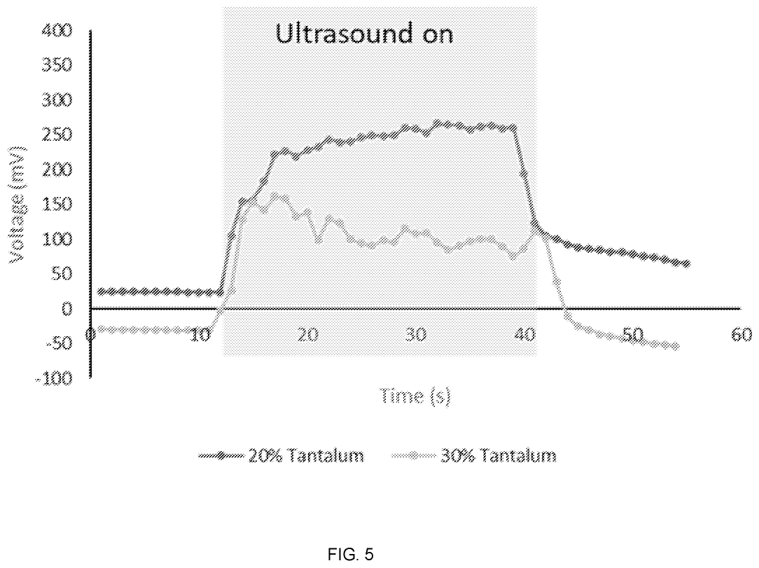

FIG. 5 is a graphical illustration of two bioelectronic compositions with varying amounts of laponite, gelatin, water, and tantalum as a contrast agent. As can be appreciated, during application of 20 kHz sonic energy, voltage was induced within the bioelectronic compositions.

In embodiments, the bioelectronic compositions described herein can be used for treatment of cancer and cancerous lesions, including but not limited to tumor ablation. In embodiments, tumor ablation is initiated by bioelectronic devices including electrodes through delivery of electrical current or charge, heating of the tumor or lesion, and application of mechanical energy, including sonic energy from ultrasound or high-intensity focused ultrasound. Typically, the standard of care for tumor treatment includes direct resection of the lesions or chemical treatments including chemotherapy. Additional therapies for tumors that are not eligible for tumor resection include thermal ablation (e.g., microwave ablation, radiofrequency ablation).

Each treatment has exposure to downsides, including damage to non-tumorous tissues surrounding the treatment area. For instance, it can be difficult for clinicians to precisely deliver ultrasound, radiofrequency, or microwave energy directly through the tumor and at therapeutic levels, given the presence of surrounding, healthy tissue or important veins and ducts (e.g. portal vein, bile duct). Delivery of an injectable bioelectronic composition can focus ablative energy towards the cancerous tissue, limiting damage of surrounding tissue. Methods include wireless electrical stimulation via external stimuli including ultrasound, high-intensity-focused ultrasound, or radiofrequency. The presence of the piezoelectric substance-based bioelectronic composition focuses these energy sources in the area of treatment by increasing the conductivity of this area or through direct flow of electrical current. Moreover, the use of the bioelectronic composition allows for the use of subthreshold energy levels, wherein the threshold is the point at which tissue damage inherently occurs.

In embodiments, the bioelectronic composition can be used for electroporation. In this treatment, the electrical stimulation induced by the piezoelectric substance-based bioelectronic composition is great enough to increase the permeability of the cell membrane. Clinical applications include treatment of benign, pre-malignant, or malignant tumors. In embodiments, this treatment can induce cell death directly through apoptosis, necrosis, necroptosis, and pyroptosis. In embodiments, this treatment is used for chemoelectroporation, a treatment which is used for patients who show no response or a poor response to typical, systemic chemotherapeutic or immune oncology drug delivery. In this iteration, the bioelectronic composition aids in chemotherapeutic or immune oncology drug delivery to the cytosol by introducing an electric current, causing easier ingress of the drug into the cell membrane. This technique can further enhance drug delivery to the brain by aiding in opening the blood brain barrier by breakdown of tight junctions and/or facilitation of transcellular passage through vesicle transport.

In embodiments, the bioelectronic composition can be used to non-invasively provide neural stimulation via electric current in sub-organ tissues. In embodiments, the bioelectronic compositions are used to target specific deep subcortical, cortical, spinal, cranial, and peripheral nerve structures to modulate neuronal activity, providing therapeutic effects for a myriad of neuropsychiatric disorders. Neural tissues of interest can include but are not limited to vagus nerve stimulation (used in the treatment of rheumatoid arthritis and Crohn's disease), splenic nerve stimulation (used in the treatment of endotoxemia), and sciatic nerve stimulation, among others. To this end, in embodiments, electrical current is generated through external stimuli (e.g., ultrasound).

In embodiments, the bioelectronic composition can be used to aid in pain management. Electrical stimulation of nerves applied transcutaneously has been used for pain management for many years. In this method, electrodes can be applied externally to introduce electrical stimulation that in turn activates a complex neuronal network to result in a reduction in pain. Given this external placement, it can be difficult to target the specific areas of pain for more precise and accurate treatment. Percutaneous electrical nerve stimulation allows for treatment of pain directly at the target treatment area. In this embodiment, the bioelectronic composition of the present disclosure is delivered percutaneously to the treatment site and external stimulation is applied for excitation of neural pathways to activate descending inhibitory systems and reduce pain.

In embodiments, the bioelectronic composition can be used to aid in wound healing. Electrical stimulation has shown promise in aiding or accelerating wound healing in chronic or diabetic wound patients. Wounds have endogenous electric fields that aid in cell migration after injury and the strength of said electric fields have been shown to enhance migration of lymphocytes, fibroblasts, macrophages, and keratinocytes. In some chronic wound cases, these electric fields are absent or perturbed, leading the patient to not respond appropriately to standard wound care. In response, researchers have explored the use of exogenous electric field stimulation through externally applied electrodes to encourage and enhance wound healing. With regard to the present disclosure, the piezoelectric substance-based bioelectronic compositions described herein can be used to introduce this exogenous electric field, stimulating cell migration and enhancing the wound healing response.

In embodiments, the bioelectronic composition can be used for cardiovascular applications. Cardiac pacemakers are a commonly used implantable bioelectronics for pacing cardiac signals using a wired electronic device. Leads of cardiac pacemakers have been linked to deleterious effects such as lack of response to cardiac resynchronization therapy, infection, fracture, and dislodgment. As a result, a need for wireless cardiac pacing technology has developed. The present disclosure describes compositions that have the capability of pacing cardiac rhythm through cyclic stimulation of the piezoelectric-based bioelectronic near to or within the cardiovascular system. The ability to deliver a bioelectronic composition within a bioelectronic device transvascularly gives the added benefit of minimal invasiveness for cardiac applications.

In embodiments, the bioelectronic composition can be used to aid in the delivery of encapsulated drugs. In embodiments, drugs are encapsulated within the bioelectronic composition, with drug release corresponding to the introduction of external stimuli including ultrasound, radiofrequency, and microwave energy. This method of drug delivery is advantageous, particularly for drugs which exhibit toxicity when delivered systemically, since drug delivery and release is targeted toward a specific treatment area. Thus, this technique can aid in delivery of chemotherapeutics without many of the deleterious effects of systemic delivery.

In embodiments, the bioelectronic compositions are the compositions described in U.S. Pat. No. 10,034,958, the contents of which are hereby incorporated by reference in its entirety. In embodiments, the bioelectronic compositions are the compositions described in U.S. Pat. No. 11,083,780, the contents of which are hereby incorporated by reference in its entirety.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

1. A piezoelectric, shear—thinning composition, comprising:
piezoelectric nanoparticles;
one or more polymers; and
deionized water.

2. The composition of claim 1, wherein the composition comprises about 0.1% to about 50% (w/w) of piezoelectric nanoparticles.

3. The composition of claim 1, wherein the piezoelectric nanoparticles are selected from the groups consisting of synthetic (laponite) and natural (bentonite, kaolinite, montmorillonite—smectite) nanoclays, quartz, zinc oxide nanoparticles, aluminum nitride.

4. The composition of claim 1, wherein the composition comprises about 0.5% to about 20% (w/w) of one or more polymers.

5. The composition of claim 1, wherein the polymer is selected from the group consisting of gelatin, collagen, chitosan, silk, polytetrafluoroethylene (PTFE), polylactic acid (PLA), poly—(l)—lactic acid (PLLA), poly (d)—lactic acid (PLDA), cellulose, alginate, agarose, starch, polyvinylidene fluoride (PVDF), polyethylene glycol (PEG), lignin, keratin, and polyvinyl alcohol (PVA).

6. The composition of claim 1, further comprising a contrast agent.

7. The composition of claim 6, wherein the contrast agent is selected from the group consisting of tantalum, tungsten, and iohexol.

8. The composition of claim 1, wherein the storage modulus (G′) of the composition is from about 1 kPa to about 40 kPa.

9. The composition of claim 1, wherein following percutaneous or vascular administration of the composition to a patient in need thereof, the exposure of the administered composition to an external stimulus provides an induced voltage from the composition.

10. The composition of claim 1, wherein the induced voltage of the composition is from about 0.01 V to about 10,000 V.

11. The composition of claim 9, wherein the external stimulus is selected from ultrasound stimulation, radiofrequency stimulation and microwave stimulation.

12. The composition of claim 11, wherein the exposure of the composition to ultrasound frequencies between about 20 kHz to about 20 MHz provides an induced voltage from the composition.

13. The compound of claim 11, wherein the exposure of the composition to radiofrequency frequencies between about between 50 MHz to about 200 MHz provides an induced voltage from the composition.

14. The compound of claim 11, wherein the exposure of the composition to microwave frequencies between about 300 MHz to about 300 GHz provides an induced voltage from the composition.

15. A plurality of piezoelectric microgels or microbeads, wherein the microgels or microbeads comprise:
piezoelectric nanoparticles; and
one or more polymers.

16. The piezoelectric microgels or microbeads of claim 15, wherein the average particle diameter of the microgels or microbeads are from about 50 microns to about 1000 microns.

17. The piezoelectric microgels or microbeads of claim 15, wherein the microgels or microbeads comprise about 0.1% to about 50% (w/w) of piezoelectric nanoparticles.

18. The piezoelectric microgels or microbeads of claim 15, wherein the piezoelectric nanoparticles are selected from the groups consisting of synthetic (laponite) and natural (bentonite, kaolinite, montmorillonite—smectite) nanoclays, quartz, zinc oxide nanoparticles, aluminum nitride.

19. The piezoelectric microgels or microbeads of claim 15, wherein the microgels or microbeads comprise about 0.5% to about 20% (w/w) of one or more polymers.

20. The piezoelectric microgels or microbeads of claim 15, wherein the polymer is selected from the group consisting of gelatin, collagen, chitosan, silk, polytetrafluoroethylene (PTFE), polylactic acid (PLA), poly—(l)—lactic acid (PLLA), poly (d)—lactic acid PLDA, cellulose, alginate, agarose, starch, polyvinylidene fluoride (PVDF), polyethylene glycol (PEG), lignin, keratin, and polyvinyl alcohol (PVA).

21. The piezoelectric microgels or microbeads of claim 15, further comprising a contrast agent.

22. The piezoelectric microgels or microbeads of claim 21, wherein the contrast agent is selected from the group consisting of tantalum, tungsten, and iohexol.

23. The piezoelectric microgels or microbeads of claim 15, wherein following percutaneous or vascular administration of the microspheres or microbeads to a patient in need thereof, the exposure of the administered composition to an external stimulus provides an induced voltage from the microspheres or microbeads.

24. The piezoelectric microgels or microbeads of claim 15, wherein the induced voltage of the microgels or microbeads is from about 0.01 V to about 10,000 V.

25. The piezoelectric microgels or microbeads of claim 23, wherein the external stimulus is selected from ultrasound stimulation, radiofrequency stimulation and microwave stimulation.

26. The piezoelectric microgels or microbeads of claim 25, wherein the exposure of the microspheres or microbeads to ultrasound frequencies between about 20 kHz to about 20 MHz provides an induced voltage from the microgels or microbeads.

27. The piezoelectric microgels or microbeads of claim 25, wherein the exposure of the microgels or microbeads to radiofrequency frequencies between about between 50 MHz to about 200 MHz provides an induced voltage from the microgels or microbeads.

28. The piezoelectric microgels or microbeads of claim 25, wherein the exposure of the microgels or microbeads to microwave frequencies between about between about 300 MHz to about 300 GHz provides an induced voltage from the microgels or microbeads.

29. A method of treating cancer or a cancerous lesion through ablation, the method comprising:
(a) administering a therapeutically effective amount of the composition of claim 1; and
(b) administering an external stimulus to provide an induced voltage from the composition.

30. The method of claim 29, wherein the composition is administered by transcatheter delivery or percutaneous injection.

31. The method of claim 29, wherein the external stimulus comprises applying sonic energy from an ultrasound or high—intensity focused ultrasound to the area where the composition is administered.

32. A method of neurostimulation, the method comprising:
(a) administering a therapeutically effective amount of the composition of claim 1; and
(b) administering an external stimulus to provide an induced voltage from the composition.

33. The method of claim 32, wherein the composition is administered by transcatheter delivery or percutaneous injection.

34. The method of claim 32, wherein the external stimulus comprises applying sonic energy from an ultrasound or high—intensity focused ultrasound to the area where the composition is administered.

35. A method of pain management, the method comprising:
(a) administering a therapeutically effective amount of the composition of claim 1; and
(b) administering an external stimulus to provide an induced voltage from the composition.

36. The method of claim 35, wherein the composition is administered by transcatheter delivery or percutaneous injection.

37. The method of claim 35, wherein the external stimulus comprises applying sonic energy from an ultrasound or high—intensity focused ultrasound to the area where the composition is administered.

38. A method of enhancing wound healing, the method comprising:
(a) administering a therapeutically effective amount of the composition of claim 1; and
(b) administering an external stimulus to provide an induced voltage from the composition.

39. The method of claim 38, wherein the composition is administered by transcatheter delivery or percutaneous injection.

40. The method of claim 38, wherein the external stimulus comprises applying sonic energy from an ultrasound or high—intensity focused ultrasound to the area where the composition is administered.

41. A method of cardiovascular pacing, the method comprising:
(a) administering a therapeutically effective amount of the composition of claim 1; and
(b) administering an external stimulus to provide an induced voltage from the composition.

42. The method of claim 41, wherein the composition is administered by transcatheter delivery or percutaneous injection.

43. The method of claim 41, wherein the external stimulus comprises applying sonic energy from an ultrasound or high—intensity focused ultrasound to the area where the composition is administered.

44. A method of electroporation, either alone or in conjunction with chemotherapeutics, the method comprising:
(a) administering a therapeutically effective amount of the composition of claim 1; and
(b) administering an external stimulus to provide an induced voltage from the composition.

45. The method of claim 44, wherein the composition is administered by transcatheter delivery or percutaneous injection.

46. The method of claim 44, wherein the external stimulus comprises applying sonic energy from an ultrasound or high—intensity focused ultrasound to the area where the composition is administered.

47. The method of claim 44, wherein the external stimulus stimulates controlled release of encapsulated therapeutic agents, including chemotherapeutic agents.
